(19) United States
(12) Patent Application Publication
(10) Pub. No.: US 2010/0030141 A1
(43) Pub. Date: Feb. 4, 2010

(54) RAPID-EXCHANGE CATHETER

(76) Inventor: Israel Chermoni, Haifa (IL)

Correspondence Address: MARTIN D. MOYNIHAN d/b/a PRTSI, INC., P.O. BOX 16446, ARLINGTON, VA, 22215, US

(21) Appl. No.: 12/448,478

(22) PCT Filed: Nov. 7, 2007
(86) PCT No.: PCT/IL07/01355

§ 371 Date: Jun. 22, 2009

(30) Foreign Application Priority Data
Dec. 20, 2006 (IL) 180221

(51) Int. Cl. A61M 25/10

(52) U.S. Cl. 604/103.04

(57) ABSTRACT

A rapid exchange catheter device comprising: an elongated collapsible catheter body with a distal end and a proximal end, having a duct along a portion of the distal end of the elongated body for engagement with a guide-wire, and having a lumen extending from the proximal end towards the distal end, a portion of the lumen at the distal end substantially parallel to at least a portion of the duct; and a stiffening insert for inserting inside the lumen up to a distal end of the lumen, so as to allow advancing the catheter by advancing the stiffening insert, which pushes the distal end of the elongated body at the distal end of the lumen, dragging the rest of the catheter behind it.

### FIELD OF THE INVENTION

The present invention relates to catheters. More particularly it relates to a rapid-exchange catheter with enhanced maneuverability.

### BACKGROUND OF THE INVENTION

Catheters are widely used in medical diagnostic and surgical procedures. In these procedures catheters are inserted in a target position in the patient's body, the target positions including, for example, blood vessels, atrium, ventricle, urethra, bladder, uterus, brain, and other body cavities.

The deployment of a catheter is an intricate procedure, involving the initial insertion of a guide-wire into the body lumen and navigating its distal end to the target location (and even some distance further beyond), after which the catheter is engaged to the guide-wire and advanced to the target location.

There are two widely used deployment methods. One is referred to as “over the wire” and the other is called “rapid-exchange”.

In the “over the wire” method the catheter is mounted over the guide-wire, the guide-wire extending inside the catheter body throughout its length. As the distal end of the catheter and the distal end of the guide-wire are located inside the body, the operator advances the catheter over the guide-wire by pushing the catheter from its proximal end, which is outside the patient's body. See, for example, U.S. Pat. No. 5,357,978 (Turk).

In the “rapid-exchange” (sometimes also called “monorail”) catheterization method the catheter is engaged to the guide-wire through a small conduit at its distal end, whereas most of the catheter's body lies adjacent the guide-wire (but not over it), so that the catheter can be quickly replaced with another (hence the name “rapid-exchange”). See, for example, U.S. Pat. No. 5,040,548 (Yock).

Typically a catheter must be flexible enough (slack-bodied) to be able to pass in tortuous passages, yet in order to successfully advance the catheter its external walls must posses some rigidity to allow the force exerted at the proximal end to pass to the distal end. This requirement means that catheters cannot be made from pliable collapsible material, and consequently a minimal threshold rigidity level cannot be passed, furthermore, as catheters are pushed from their proximal end, it is imperative that the catheter-body possesses some rigidity or else it would have been impossible to advance a catheter forward at all.

In order to overcome the problem of advancing slack bodied catheters several deployment methods and devices were introduced.

U.S. Pat. No. 5,578,009 (Kraus et al.), disclosed an elongated rod, terminating in an abutment device with a lumen for passing of a guidewire, used to push an angioplasty balloon along the guidewire.

In U.S. Pat. No. 6,254,610 (Darvish et al.) a device for dragging and positioning of a catheter within a duct in a body was disclosed comprising a rod whose distal end distal end is coupled to a loop, designed to be slidably mounted over a guide-wire, and provided with hooking means, onto which catheter is hooked, so that when advancing the rod from its distal end, the loop slides forward over the guidewire, and drags the catheter to its target location. In this patent the catheter is dragged from its distal end rather pushed from behind.

US 2005/028870 (Chermoni) discloses a catheter with a hydraulic fluid column adapted to apply a pushing force to the front tip of the catheter. Optionally, the body of the catheter comprises two tubes, one or which extends in response to the pushing force (see also WO2005115524).

It is a purpose of the present invention to provide a catheter of novel design possessing “rapid-exchange” quality and very high flexibility.

Another purpose of the present invention is to provide a catheter with modifiable rigidity thus allowing the use of the same catheter is cavities of different properties and characteristics. This way the range of different uses and tasks of the same catheter are greatly expanded.

Other advantages and objects will become apparent after reading the present specification and considering the accompanying figures.

### SUMMARY OF THE INVENTION

There is thus provided, in accordance with a preferred embodiment of the present invention, a rapid exchange catheter device comprising:

an elongated collapsible catheter body with a distal end and a proximal end, having a duct along a portion of the distal end of the elongated body for engagement with a guide-wire, and having a lumen extending from the proximal end towards the distal end, a portion of the lumen at the distal end substantially parallel to at least a portion of the duct; and a stiffening insert for inserting inside the lumen up to a distal end of the lumen, so as to allow advancing the catheter by advancing the stiffening insert, which pushes the distal end of the elongated body at the distal end of the lumen, dragging the rest of the catheter behind it.

Furthermore, in accordance with some preferred embodiments of the present invention, the stiffening insert is made from metal.

Furthermore, in accordance with some preferred embodiments of the present invention, the stiffening insert is made from steel.

Furthermore, in accordance with some preferred embodiments of the present invention, the stiffening insert has a varying stiffness.

Furthermore, in accordance with some preferred embodiments of the present invention, the stiffening insert has a continuously varying stiffness.

Furthermore, in accordance with some preferred embodiments of the present invention, the stiffening insert has a discretely varying stiffness.

Furthermore, in accordance with some preferred embodiments of the present invention, the stiffening insert has a varying cross-section.

Furthermore, in accordance with some preferred embodiments of the present invention, the insert has a continuously varying cross-section.

Furthermore, in accordance with some preferred embodiments of the present invention, the insert has a discretely varying cross-section.

Furthermore, in accordance with some preferred embodiments of the present invention, the device comprising a balloon.

Furthermore, in accordance with some preferred embodiments of the present invention, the distal end of the elongated body comprises a catheter head, wherein the distal end of the lumen is fluidically connected to an annular void provided about the catheter head, a balloon wall externally covering the void.

Furthermore, in accordance with some preferred embodiments of the present invention, the annular void is fluidically connected to an inner space through at least one bore.

Furthermore, in accordance with some preferred embodiments of the present invention, the balloon wall is attached to the catheter head at ends under which a slit is provided, substantially parallel to the balloon wall.

Furthermore, in accordance with some preferred embodiments of the present invention, a distal end of the stiffening insert is engaged at the distal end of the lumen to the distal end of the catheter.

Furthermore, in accordance with some preferred embodiments of the present invention, the stiffening insert is removable from the catheter.

Furthermore, in accordance with some preferred embodiments of the present invention, there is provided a method for rapid exchange catheterization comprising:

providing an elongated collapsible catheter body with a distal end and a proximal end, having a duct along a portion of the distal end of the elongated body for engagement with a guide-wire, and having a lumen extending from the proximal end towards the distal end, a portion of the lumen at the distal end substantially parallel to at least a portion of the duct; and a stiffening insert for inserting inside the lumen up to a distal end of the lumen;

advancing the catheter by advancing the stiffening insert, which pushes the distal end of the elongated body at the distal end of the lumen, dragging the rest of the catheter behind it.

Furthermore, in accordance with some preferred embodiments of the present invention, the method further comprises removing the stiffening insert.

Furthermore, in accordance with some preferred embodiments of the present invention, the method further comprises replacing the stiffening insert with a stiffening insert having a different stiffness characteristic than the stiffness characteristic of the replaced stiffening insert.

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1 illustrates a sectional view of an improved rapid-exchange catheter according to a preferred embodiment of the present invention, for angioplasty uses, inserted in a body lumen.

FIG. 2a illustrates a sectional view of an improved rapid-exchange catheter according to another preferred embodiment of the present invention, for angioplasty uses, inserted in a body lumen.

FIG. 2b illustrates sectioned detail of the catheter shown in FIG. 2a—its front (distal) end.

FIG. 3a illustrates a general view of an improved rapid-exchange catheter according to a preferred embodiment of the present invention.

FIG. 3b illustrates a sectional view of the catheter shown in FIG. 3a, inserted in a body lumen.

FIG. 3c illustrates the rear (proximal) end of the catheter shown in FIG. 3b.

FIG. 4a illustrates a sectional view of an improved rapid-exchange catheter according to another preferred embodiment of the present invention, for angioplasty uses.

FIG. 4b illustrates a stiffening insert with continuously varying stiffness along its body, for incorporation with an improved rapid-exchange catheter, according to a preferred embodiment of the present invention.

FIG. 4c illustrates a stiffening insert with discretely varying stiffness along its body, for incorporation with an improved rapid-exchange catheter, according to a preferred embodiment of the present invention.

FIG. 5 illustrates holding the proximal end of an improved rapid-exchange catheter according to a preferred embodiment of the present invention and advancing it.

### DETAILED DESCRIPTION OF PREFERRED EMBODIMENTS

A main concept of the present invention is the provision of an improved rapid-exchange catheter, which is advanced to its target position by way of applying a pulling force on its front distal end, instead of pushing it from behind (its proximal end). In order to facilitate this a stiffening insert is introduced into the body of the catheter, whose distal end is either engaged to or leans against a location at the distal end of the catheter and which when pushing the distal end of the catheter forward causes the body of the catheter with its entire length to be dragged behind the distal end. This eliminates the need for a stiff catheter body allowing the catheter body to be made from extremely pliable collapsible material, as there is no longer a need to transfer a pushing force exerted at the proximal end of the catheter along the catheter body all the way to its distal end in order to advance it forward.

The wall of the catheter tube can be collapsible but not stretchable, as it has to transfer liquid pressure into the balloon at the catheter head.

Another main aspect of the present invention stems from the understanding that in order to facilitate the existence of highly flexible catheters their deployment ought to be carried out regardless the rigidity presented by the catheter body itself, rather use an stiffening insert that is engaged to the distal end of the catheter and passes through the body of the catheter. When advancing the catheter, the stiffening insert is pushed its distal end pushing against the distal end of the catheter from within, thus effectively dragging the catheter body behind the distal end.

The stiffening insert is preferably in the form of a wire insert made of a material substantially harder than the body of the catheter (typically steel, preferably steel 316, which is bio-compatible, but other materials can be used too).

In some preferred embodiments of the present invention the stiffening insert is provided in several diameters in order to facilitate modifiable rigidity, but replacing one stiffening insert with another of different diameter. This is useful when the catheterization procedure is halted as a result of excessive friction, or when the operator realizes that the friction inside the body is very low. In this case the operator can replace the stiffening insert with another stiffening insert of lower rigidity (smaller diameter) in the case of excessive friction, or higher rigidity (larger diameter) in the case of very low friction.

In some embodiments of the present invention the stiffening insert has varied stiffness along its length. This is to compensate for the fact that as the catheter is advanced into the body it becomes increasingly harder to advance it further due to friction exerted by the increasingly longer path it takes. In order to overcome this problem the stiffening insert is softer at its distal end and becomes increasingly harder further down its length towards the proximal end.

The stiffness of the stiffening insert may vary in a continuous manner or discretely, or may be fixed along the entire body of the stiffening insert.

During a angioplasty catheterization procedure, the catheter may be positioned within an occlusion, so that more force is needed to expand the volume of the balloon as the occlusion resists the expansion of the balloon. In this case the stiffening insert is withdrawn, the catheter itself remaining in position due to the friction exerted by the lumen wall on the catheter head, allowing more liquid volume to be pumped into the catheter head as a result of the free space available due to the removal of the insert.

Reference is now made to the figures.

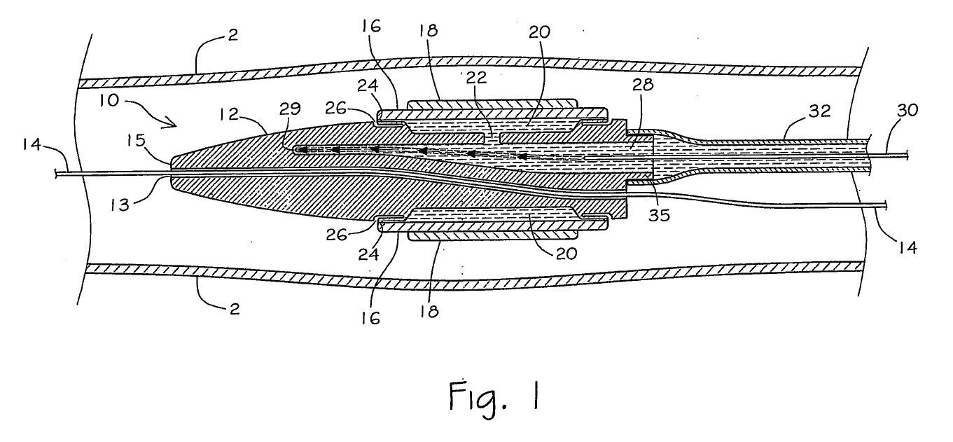

FIG. 1 illustrates a sectional view of an improved rapid-exchange catheter according to. a preferred embodiment of the present invention, for angioplasty uses, inserted in a body lumen.

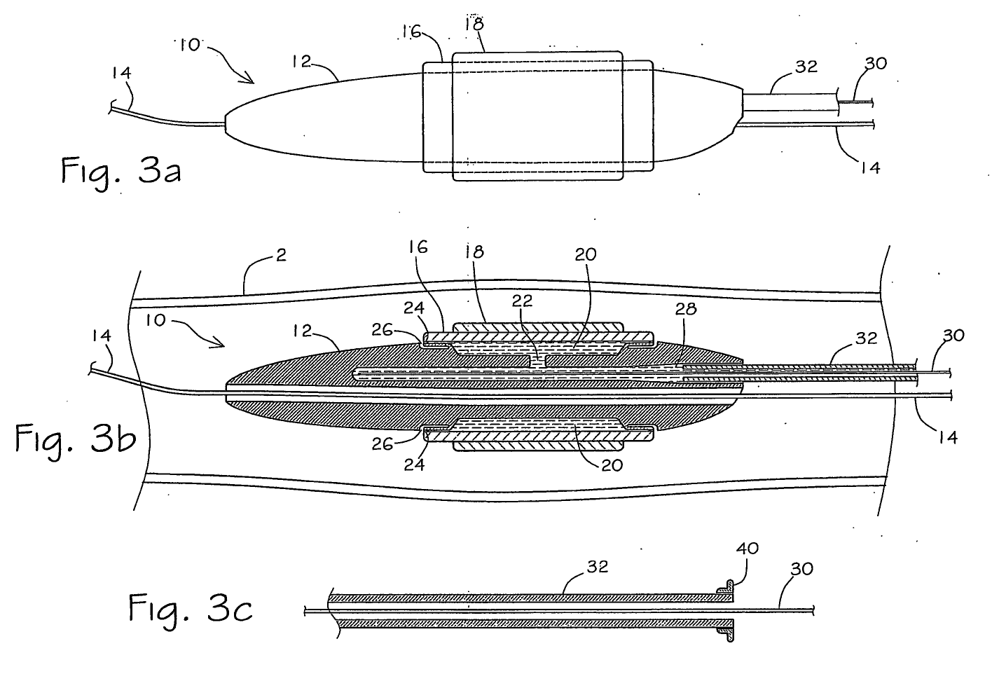

Shown is the distal portion of the catheter, which goes into the body lumen and is aimed at being deployed at a desired target location within the lumen 2. The catheter 10 shown is an angioplasty catheter for insertion into the vasculature and for performing dilation where an occlusion occurs and for deployment of a stent. It comprises a catheter head 12 through which a duct 13 is provided for a guide-wire 14 to pass through. As this is a rapid exchange catheter the guide-wire duct crosses the catheter head 12 but not further the length of the catheter tube 32. Catheter tube 32 leads liquid through its lumen into the catheter head 12 into a cavity 28 inside the catheter head, which extends towards the front distal end of the head up to point 29. Stiffening insert 30 is inserted into the catheter along its tube 32 and into cavity 28 of the head 12, and is advanced (the arrows indicating the direction of advance) until it reaches point 29 (see also FIG. 3b). From that moment any additional forward motion by the stiffening insert will result in exertion of a pushing force on the inside of distal point 29 of the catheter head 12, which leads to the catheter head 12 being advanced forward, dragged along almost all of its entire length (apart from a small portion at the front of the catheter head which lies between point 29 and the very distal tip 15 of the head. When the catheter head has reached its final destination, stiffening insert 30 may be withdrawn leaving the catheter head in position. Alternatively the stiffening insert may remain inside.

The catheter head has a novel design. External balloon wall 16, on which stent 18 may be further provided, encloses void 20, which extends annularly about the catheter head and which is fluidically connected through at least one bore 22 to cavity 28. The balloon wall 16 is attached at its ends 24 to the catheter head, and small cuts 26 are provided in the catheter head, substantially parallel to the end 24 of balloon wall 16. When liquid is forced into the catheter head the internal pressure rises causing the attached ends 24 of balloon wall 16 to rise outwardly expanding balloon wall outwardly and consequently forcing stent 18 open.

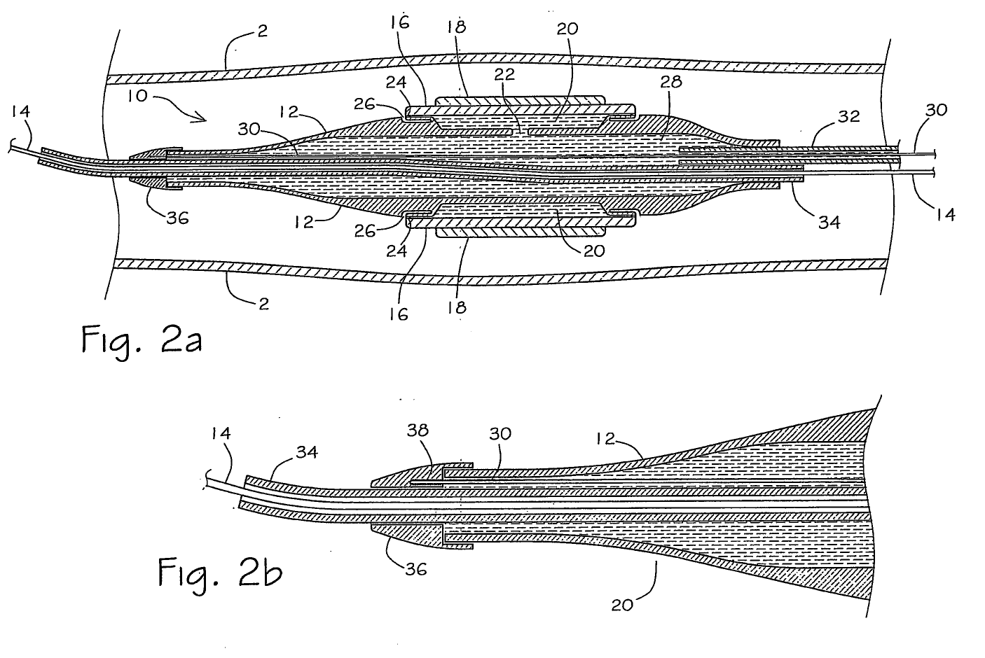

FIG. 2a illustrates a sectional view of an improved rapid-exchange catheter according to another preferred embodiment of the present invention, for angioplasty uses, inserted in a body lumen. This is a slight modification of the catheter shown in FIG. 1. The catheter head 12 is provided with cap 36, which is secured to the distal end of the catheter head 12. A short tube 32 is provided inside duct 13 for stiffening the duct through which guide-wire 14 passes, the end of the short tube passing through cap 36.

FIG. 2b illustrates sectioned detail of the catheter shown in FIG. 2a—its front (distal) end. Cap 36 is provided with a slit 38 into which the distal end of stiffening insert 30 may be brought. In this catheter version the stiffening insert 30 reaches to the very distal end of the catheter head 12, so practically the entire length of the catheter as well as its head are dragged behind the distal end of the stiffening insert as it is advanced forward. Slit 38 may be narrow enough to firmly hold the distal end of the stiffening insert preventing its removal, or it may be wide enough for the distal end of the stiffening insert to be inserted and removed at will.

FIG. 3a. illustrates a general view of an improved rapid-exchange catheter according to a preferred embodiment of the present invention. Tube 32 may be separate from the catheter head and plugged into the catheter head using a female connection as shown in this figure (and in FIG. 2a). Alternatively it may be plugged to the catheter head over a male connection—flange 35 (see FIG. 1).

FIG. 3b illustrates a sectional view of the catheter shown in FIG. 3a, inserted in a body lumen.

FIG. 3c illustrates the proximal end of the catheter shown in FIG. 3b. The stiffening insert 30 is inserted into tube 32 of the catheter through flange 40 provided at the proximal end of the catheter tube.

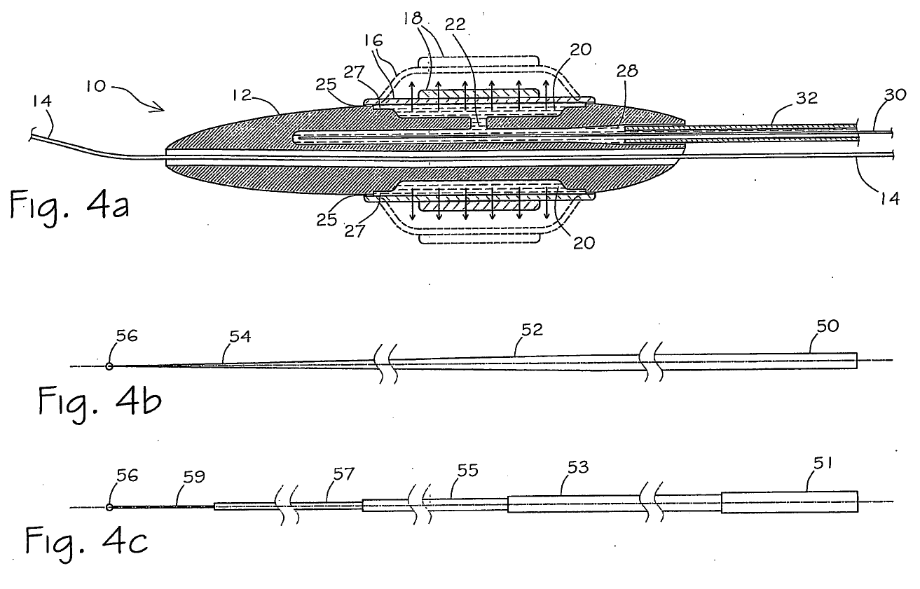

FIG. 4a illustrates a sectional view of an improved rapid-exchange catheter according to another preferred embodiment of the present invention, for angioplasty uses, in two states—deflated and inflated. Ends 25 of balloon wall 16 are attached to, the body of catheter head 12 over annular void 20, with optional slits 27 beneath ends 25 cut into the body of catheter head 12. When liquid is forced into cavity 28 of catheter head 12, pressure is increased in annular void 20 causing balloon wall 16 to bulge outwardly, deploying stent 18.

FIG. 4b illustrates a stiffening insert with continuously varying stiffness along its body, for incorporation with an improved rapid-exchange catheter, according to a preferred embodiment of the present invention. This is achieved by varying the diameter (cross-section) of the insert continuously along its length.

The varying stiffness has a significant appeal in the context of the present invention, as distal tip 56, which travels deepest into the body lumen has nothing in front of it (no additional insert material) to push forward and on the other hand has to take up the form of any turn or bent it comes across in order to advance properly. Moreover, it has to be guided and navigated to the target location and for these ends it must be rather flexible. At point 54, which is near the distal tip yet behind point 56 there is a need for greater rigidity, as at this point the front portion of the stiffness insert (up to tip 56) needs to be pushed forward. Yet greater rigidity is required at point 52 further down stiffening insert 30, and still greater rigidity is required at proximal end 50 of the stiffening insert. As we proceed further down the stiffening insert there is less need for flexibility (for a greater portion of the insert has already advanced in front) and greater need for rigidity as more insert material has to be pushed forward.

FIG. 4c illustrates a stiffening insert with discretely varying stiffness along its body, for incorporation with an improved rapid-exchange catheter, according to a preferred embodiment of the present invention. Here the rigidity (flexibility) of the stiffening insert varies discretely, with segments of different rigidity 59, 57, 55, 53, 51—each segment of having a fixed diameter throughout the segment. This is achieved by varying the diameter (cross-section) of the insert discretely along its length.

The stiffening insert can possess a varying stiffness not only by changing its diameter along its length. The stiffening insert may be made from materials with varying density, or from different materials placed in different parts along the length of the insert, the different materials having different stiffness characteristics.

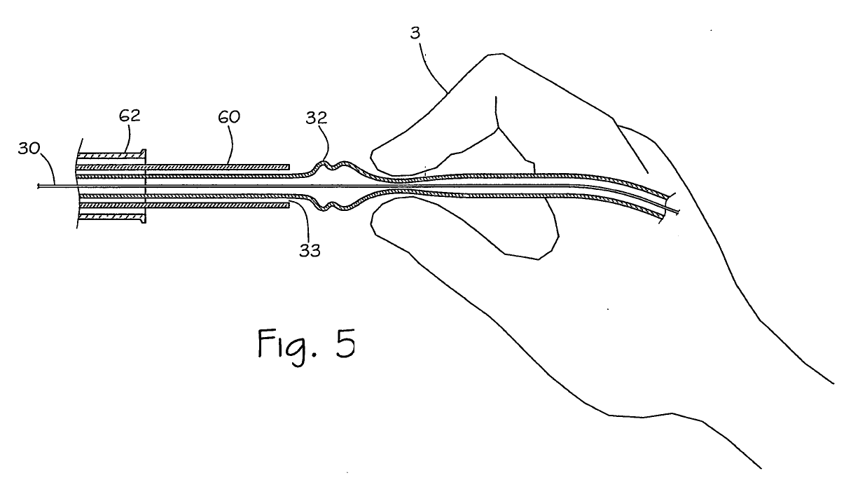

FIG. 5 illustrates holding the proximal end of an improved rapid-exchange catheter according to a preferred embodiment of the present invention and advancing it.

It is evident that when the proximal portion of the catheter tube 32 is held firmly between fingers 31 of the operator and advanced into the guide catheter 60, which is inserted in an Easy-catch™ (by Superior Healthcare Group) applicator 62, tube wall 32, which is collapsible, becomes wrinkled and slack, whereas stiffening insert transfers the force from the operator's hand towards the distal tip of the stiffening insert. The body of the catheter is dragged behind its distal tip.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the present invention.

1. A catheter device comprising:
an elongated collapsible catheter body with
a distal end and a proximal end,
a balloon at said distal end, and
having a lumen extending from the proximal end towards the distal end, a portion of the lumen at the distal end extending past a proximal end of said balloon; and
a stiffening insert for inserting inside the lumen up to a distal end of the lumen, so as to allow advancing the catheter by advancing the stiffening insert, which pushes the distal end of the elongated body at the distal end of the lumen, dragging the rest of the catheter behind it.

2. The device as claimed in claim 1, wherein the stiffening insert is made from metal.

3. The device as claimed in claim 2, wherein the stiffening insert is made from steel.

4. The device as claimed in claim 1, wherein the stiffening insert has a varying stiffness.

5. The device as claimed in claim 4, wherein the stiffening insert has a continuously varying stiffness.

6. The device as claimed in claim 4, wherein the stiffening insert has a discretely varying stiffness.

7. The device as claimed in claim 4, wherein the stiffening insert has a varying cross-section.

8. The device as claimed in claim 7, wherein the insert has a continuously varying cross-section.

9. The device as claimed in claim 7, wherein the insert has a discretely varying cross-section.

10. The device as claimed in claim 1, wherein said lumen is fluidically connected to said balloon.

11. The device as claimed in claim 10, wherein the distal end of the elongated body comprises a catheter head, wherein the distal end of the lumen is fluidically connected to an annular void provided about the catheter head, a balloon wall externally covering the void.

12. The device as claimed in claim 11, wherein the annular void is fluidically connected to an inner space through at least one bore.

13. The device as claimed in claim 11, wherein the balloon wall is attached to the catheter head at ends under which a slit is provided, substantially parallel to the balloon wall.

14. The device as claimed in claim 1, wherein a distal end of the stiffening insert is engaged at the distal end of the lumen to the distal end of the catheter.

15. The device as claimed in claim 1, wherein the stiffening insert is removable from the catheter.

16. A method for catheterization comprising:
inserting a stiffening insert into a catheter with a balloon section at a distal end thereof, past a proximal side of said balloon section;
and
advancing the catheter by advancing the stiffening insert, from a proximal end thereof, which pushes the distal end of the elongated body at the distal end of the lumen, dragging at least part of the catheter behind balloon section.

17. The method of claim 16, further comprising removing the stiffening insert.

18. The method of claim 16, further comprising replacing the stiffening insert with a stiffening insert having a different stiffness characteristic than the stiffness characteristic of the replaced, stiffening insert.

19. The method as claimed in claim 16, wherein the stiffening insert has a varying stiffness.

20. The method as claimed in claim 19, wherein the stiffening insert has a continuously varying stiffness.

21. The method as claimed in claim 19, wherein the stiffening insert has a discretely varying stiffness.

22-23. (canceled)

24. A method according to claim 16, comprising mechanically coupling said stiffening element to a proximal side of said catheter.

25. A method according to claim 16, wherein said advancing comprises advancing by applying force from a proximal side of said catheter, bypassing a part of said catheter and applying said force at said distal end of said catheter.

26. A method according to claim 16, wherein said advancing comprises advancing causing a part of said catheter to be slack during said advancing.

27. A catheter device according to claim 1, wherein said device is a rapid exchange catheter device.

28. A catheter device according to claim 1, wherein said body has a duct along a portion of the distal end of the elongated body for engagement with a guide-wire substantially parallel to at least a portion of the lumen.

29. A catheter device according to claim 1, wherein said device is pliable absent said stiffening insert.

30. A catheter device according to claim 1, wherein a proximal part of said catheter is adapted for engaging said stiffening insert.

31. A catheter device according to claim 1, wherein said lumen is sealed at a distal end thereof.

32. A catheter device according to claim 1, wherein said lumen extends past a distal end of said balloon.
